(19) United States
(12) Patent Application Publication
(10) Pub. No.: US 2009/0306605 A1
(43) Pub. Date: Dec. 10, 2009

(54) Medical Treatment Device and Method for Manufacturing the Same

(76) Inventors: Kenichi Amano, Shizuoka (JP); Tomokazu Sano, Shizuoka (JP); Kyoko Nishitani, Shizuoka (JP)

Correspondence Address: Carter, DeLuca, Farrell & Schmidt, 445 Broad Hollow Road, Suite 225, Melville, NY, 11747, US

(21) Appl. No.: 11/885,307

(22) PCT Filed: Mar. 3, 2005
(86) PCT No.: PCT/EP05/02258

§ 371 Date: Aug. 6, 2009

(51) Int. Cl. A61M 25/00; B05D 3/00; B05D 3/02; B05D 3/06

(52) U.S. Cl. 604/265; 427/2.3

(57) ABSTRACT

To provide a medical treatment device wherein the antithrombotic property and lubricity of a coating formed on the surface can be maintained in a stable state for a long period and also to provide a method for manufacturing the same. A mixture solution for coating wherein a copolymer of methyl vinyl ether with maleic acid anhydride, a quaternary ammonium salt and a polyether block amide dissolved in an organic solvent are applied upon the surface of a substrate comprising synthetic resin to form a coating and then urokinase and heparin are impregnated in the coating of the medical treatment device. The synthetic resin comprising the substrate is polyurethane, polyvinyl chloride, nylon or nylon elastomer. The quaternary ammonium salt is tridodecylmethylammonium chloride. An alkaline treatment is applied to a predetermined part of a substrate upon which a coating has been formed by application of the mixture solution.

The present invention relates to a medical treatment device wherein a coating having an antithrombotic property and lubricity is formed on the surface of a catheter, for example, and also a method for manufacturing the same.

### BACKGROUND ART

Among medical treatment devices are those which are punctured into and left to remain in blood vessels such as catheters or guide needles used by inserting into a catheter. Since such medical treatment devices are foreign substances to the blood, when the medical treatment device makes contact with blood in blood vessel, a blood coagulation reaction may take place and a thrombus block may be formed. As a result of such formation of thrombus blocks, there are some cases in which not only is the original objective, treatment or diagnosis of diseases, not achieved but new complications are generated by the thrombus which may be severe. Therefore, antithrombotic property is required for the medical device contacting blood in the blood vessel. a method whereby a coating of a copolymer of methyl vinyl ether with maleic acid anhydride which is to be a binder for polyurethane and urokinase is formed on the surface of a substrate comprising polyurethane and then urokinase is contained in the coating by means of impregnation or the like (refer, for example, to Patent Document 1). In addition, lubricity is required of such a medical treatment device for preventing damage to mucous membranes or for attenuating pain in patients when the device is inserted into the body. With regard to such medical treatment devices having lubricity, there is a product in which a fibrinolysis activating substance is bonded to a cross-linked coating comprising polyol and polymer having an acid anhydride group whereby lubricity is expressed upon being moisturized (refer, for example, to Patent Document 2).

Patent Document 1: Japanese Patent Laid-Open No.

Patent Document 2: Japanese Patent Laid-Open No.

### DISCLOSURE OF THE INVENTION

Among the medical treatment devices as mentioned above, there is a catheter which is used in a state of being retained in a blood vessel of a patient for as long as one month. However, a coating for expressing an antithrombotic property in the above-mentioned conventional medical treatment device has insufficient durability and could not be used for a long period. In addition, lubricity could not be maintained for a long period.

The present invention has been achieved in view of such circumstances and its object is to provide a medical treatment device wherein the antithrombotic property and lubricity of a coating formed on the surface can be maintained in a stable state for a long period and also to provide a method for manufacturing the same.

In order to achieve the above-mentioned object, characteristic features of the medical treatment device concerning the present invention are that a fibrinolysis activating enzyme fixer for fixation of fibrinolysis activating enzyme, a suppressor fixer for fixation of suppressor which suppresses blood coagulation factor activity and a fixing reinforcer for reinforcing fixing action of the fibrinolysis activating enzyme fixer and the suppressor fixer, are dissolved in an organic solvent to manufacture a mixture solution for coating, the mixture solution for coating is applied on the surface of a substrate comprising synthetic resin to form a coating and, at the same time, the fibrinolysis activating enzyme, the suppressor or both the fibrinolysis activating enzyme and the suppressor are combined with the coating.

In the thus constituted medical treatment device, the coating formed on the surface of the substrate contains a fibrinolysis activating enzyme bonded to the substrate by a fibrinolysis activating enzyme fixer, a suppressor which is bonded to the substrate by a suppressor fixer or both the fibrinolysis activating enzyme and the suppressor. Both the fibrinolysis activating enzyme and the suppressor have an antithrombotic property. In addition, the coating contains a fixation promoter which enhances the durability of the antithrombotic property by strengthening the close contact of both the fibrinolysis activating enzyme fixer and the suppressor fixer to the coating. Accordingly, in the medical treatment device of the present invention, a synergistic effect is achieved due to the use of both a fibrinolysis activating enzyme and a suppressor whereby far better antithrombotic property and durability are attained compared to the antithrombotic property and durability of the medical device having a coating containing either the fibrinolysis activating enzyme or the suppressor but not both. Incidentally, even when either the fibrinolysis activating enzyme or the suppressor is used but not both together, an excellent effect can be achieved as well.

Another compositional characteristic of the medical treatment device according to the present invention is that an alkaline treatment comprising impregnation in an alkaline solution is applied to a predetermined part of a substrate on which a coating has been formed by application of the mixture solution for coating. This alkaline treatment of a coating formed on a substrate, on the surface of the medical treatment device, is carried out so that lubricity is generated when the device is moisturized. The alkaline treatment at that time is conducted by impregnation of the formed coating in an alkaline solution such as sodium hydroxide or potassium hydroxide for a predetermined time. The alkaline treatment need be applied only to the part of the medical treatment device which needs to have lubricity. For example, when the medical treatment device is a catheter, the treatment may be conducted to all of the parts which are inserted into body or may be conducted to the leading end part only.

With regard to a fixer for a fibrinolysis activating enzyme, a copolymer of methyl vinyl ether with maleic acid anhydride may be used while, with regard to a fixing reinforcer for strengthening refixing action, a polyether block amide may be used. The suppressor fixer may be a quaternary ammonium salt, and the quaternary ammonium salt may be tridodecylmethylammonium chloride. Since, among the quaternary ammonium salts tridodecylmethylammonium chloride is characterized by excellent close adhesion to synthetic resins, it is able to achieve a particularly pronounced effect.

The fibrinolysis activating enzyme may be urokinase, streptokinase, tissue plasminogen activator, plasmin or prinolase. The suppressor may be heparin, hirudin, thrombomodulin or an antiplatelet substance.

Still another constitutional characteristic of the medical treatment device according to the present invention is that the synthetic resin constituting the substrate is polyurethane, polyvinyl chloride, nylon or nylon elastomer. As a result, affinity between the substrate and the fixing reinforcer for strengthening the fixing agent, such as polyether block amide, increases, whereby the antithrombotic property and durability of the formed coating are improved.

A compositional characteristic of a method for the manufacture of a medical treatment device according to the present invention is that it comprises a step for the manufacture of a mixture solution for coating in which a fibrinolysis activating enzyme fixer for fixation of fibrinolysis activating enzyme, a suppressor fixer for fixation of suppressor which suppresses blood coagulation factor activity and a fixing reinforcer for strengthening fixing action of the fibrinolysis activating enzyme fixer and the suppressor fixer are dissolved in an organic solvent, a step for the formation of a coating in which the mixture solution for coating is applied on the surface of a substrate comprising a synthetic resin to form a coating and a step for impregnation in which the fibrinolysis activating enzyme, the suppressor or both the fibrinolysis activating enzyme and the suppressor are impregnated in the coating.

After the coating formation step, it is also possible to add an alkaline treatment step wherein a predetermined part of the substrate whereupon the coating is formed is immersed in an alkaline solution. As a result, not only an antithrombotic property but also lubricity can be achieved in the medical treatment device. In such a case, a copolymer of methyl vinyl ether with maleic acid anhydride may be used as a fixer for fibrinolysis activating enzyme, a quaternary ammonium salt may be used as a fixer for suppressor, a polyether block amide may be used as a fixing reinforcer for fixing action, urokinase may be used as a fibrinolysis activating enzyme and heparin may be used as a suppressor. As a result, a coating having an antithrombotic property on the substrate surface can be formed in a stable manner by a simple treatment step which thus can be applied to actual mass-production.

Another constitutional characteristic of the medical devices according to the present invention is that, after the coating step formation, there is provided a drying step where the coating is subjected to a drying treatment at a temperature range from room temperature to 80° C. As a result, a coating having sufficient and stable antithrombotic property can be formed by a simple treatment step which in addition can be applied to various medical treatment devices which are deformed by a high temperature treatment, such as catheters.

Another constitutional characteristic of the method for the manufacture of medical treatment device according to the present invention is that, after the step for impregnation, there is provided a sterilizing step where the coating is sterilized by gamma radiation. The sterilization by gamma radiation acts upon molecules of the substance directly, without utilization of heat and chemical reaction and, therefore, safe and sure sterilization can be efficiently carried out. As a result, urokinase can be fixed to the medical treatment device while sufficiently retaining the effectiveness of the urokinase, so that it is possible to manufacture a safer medical treatment device as well.

### BEST MODE FOR CARRYING OUT THE INVENTION

In the medical treatment device according to the present invention, a substrate comprising a synthetic resin is immersed in a mixture solution for coating which is prepared by dissolving a copolymer of methyl vinyl ether with maleic acid anhydride, a quaternary ammonium salt and a polyether block amide in an organic solvent, whereby a coating is formed on the surface of the substrate. When the substrate upon which the coating is formed is immersed in an aqueous solution containing urokinase and heparin, urokinase and heparin is incorporated into the coating.

In this coating, urokinase and heparin are present for a long period and antithrombotic property and durability thereof are effectively achieved. Also, after a coating is formed on the substrate surface, a predetermined part of the coating can be immersed in an alkaline solution for a predetermined time to conduct an alkaline treatment. As a result, lubricity can be given to the coating. Antithrombotic property, lubricity and durability thereof in the medical treatment device prepared as such will be illustrated in the following Examples 1 and 2.

### Example 1

Tridodecylmethylammonium chloride (trade name: Tridodecylmethylammonium Chloride, manufactured by Polysciences; hereinafter referred to as “C”) was added to a mixture solution wherein a 2% solution of a copolymer of methyl vinyl ether with maleic acid anhydride (trade name: Gantrez AN-169, manufactured by ISP (International Specialty Products); hereinafter referred to as “A”) in acetone was mixed with a 2% solution of a polyether block amide (trade name: Pebax 2533 SA, manufactured by Atochem; hereinafter referred to as “B”) in THF in a ratio of 1.5:1, whereby A:B:C was made 3:2:1, to prepare a mixture solution for coating.

A substrate was immersed in this mixture solution for coating, removed, dried under reduced pressure for 3 hours at a drying temperature of 60° C., immersed at 5° C. for 24 hours into a 0.7% solution of heparin sodium (manufactured by Diosynth) mixed in acidic physiological saline (pH=4.6) containing 300 IU/ml of urokinase (manufactured by JCR), removed and dried in vacuo to form a coating on the surface of the substrate. The substrate surface including the coating was irradiated with 40 kGY of gamma radiation for sterilization.

The substrate used was a tube made of polyurethane (trade name: Tecoflex; manufactured by Thermedics), of which the diameter was 14G and the full length was 20 cm. The coating formed in the inner area of the tube was subjected to the following antithrombotic test. The same antithrombotic test was carried out for the coating formed on the inner coating of a medical treatment device according to the Comparative Examples and also for the tube itself. The result of those tests is shown in FIG. 1.

In the antithrombotic test, an aqueous solution of Na⁺ and Ca²⁺ in the same concentrations as in plasma (wherein 8,307 mg of NaCl and 278 mg of CaCl₂ were dissolved in 1 liter of distilled water) and kept at 37° C., the same as body temperature was prepared. The aqueous solution was passed into an inner area of the tube at a flow rate of 20 ml/hour for 0, 1, 5, 10, 15, 20, 25 or 30 days to rinse the coating formed in the inner area. After that, 0.1 ml of human whole blood was placed into the inner area of the tube at room temperature and both ends of the tube was connected and sealed to form a loop.

The tube loop was rotated at the rate of 5 r.p.m. and the time it took for the blood to lose fluidity was measured as equivalent to the time for thrombus generation. In this operation, while the blood remained at the lower side of the rotating tube loop, it was judged that the blood had fluidity, and when blood began to rotate together with the tube loop, it was judged that fluidity was lost. For determining each rinsing time, n=5 data on the whole blood of five persons was averaged.

In FIG. 1, the abscissa shows the period of rinsing (day(s)) and the ordinate shows time (hour(s)) for generation of thrombus. In Comparative Examples 1 and 2, a commercially available product wherein urokinase was fixed on the surface of the tube was used and, in Comparative Example 3, the same wherein heparin sodium was fixed on the surface of the tube was used. In Comparative Example 4, a product wherein no coating had been formed on the surface of the tube was used.

As will be apparent from the test result shown in FIG. 1, in the case of Example 1, blood retained fluidity and no thrombus was generated even after 4 hours from the initiation of the test in all cases in which the rinsing period was from 0 to 30 day(s). On the contrary, in Comparative Examples 1 to 3, blood retained fluidity and no thrombus was generated even after 4 hours from the initiation of the test when the rinsing period was 0 to 5 day(s) while, when the rinsing period was 10 to 30 days, blood lost fluidity and thrombus was generated within 4 hours from initiation of the test. In addition, in these cases, there was noted the tendency that the longer the rinsing period, the shorter the thrombus generation time. In Comparative Example 4, thrombus was generated within 20 minutes from the initiation of the test in all cases in which the rinsing period was from 0 to 30 day(s).

From these results, it is concluded that, in Example 1, effective amounts of urokinase and heparin remained in the coating whereby the antithrombotic property was maintained in any of the cases in which the rinsing period was 0 to 30 day(s). In Comparative Examples 1 to 3, it was presumed that effective amounts of urokinase and heparin remained in the coating, that is, antithrombotic property was maintained, when the rinsing period was 1 to 5 day(s) while, when the rinsing period was 10 to 30 days, it was concluded that an effective amount of urokinase or heparin did not remain in the coating. On the basis of these results, it is presumed that the longer the rinsing period, the less the residual amounts of urokinase and heparin.

From the above results, it may be concluded that the medical treatment device according to Example 1 is superior to any of the medical treatment devices of Comparative Examples 1 to 4 in the degree and durability of antithrombotic property. That is because both urokinase and heparin are fixed to the substrate by a methyl vinyl ether/maleic acid anhydride copolymer and tridodecylmethylammonium chloride, respectively, and further because the fixation is reinforced by a polyether block amide. As a result, it is now possible to prepare a medical treatment device wherein a durable antithrombotic property of the surface is achieved.

### Example 2

The substrate in the step for the manufacture of the test sample for Example 1 was immersed in a mixture solution for coating, dried under reduced pressure at 60° C. for 3 hours and subjected to an alkaline treatment wherein the lubricated part of the substrate was immersed for 3 minutes in a 0.1N aqueous solution of sodium hydroxide. After the substrate was removed from the sodium hydroxide solution, it was dried under reduced pressure at 60° C. for 3 hours. All other steps were the same as those in the manufacture of the test sample for Example 1 to prepare the test sample which will be called Example 2. The test for surface lubricity was conducted for the test samples of Example 2 and the above-mentioned Comparative Examples 1 to 4. The result is shown in the following Table 1.

TABLE 1

| | | Lubricity | Lubricity after |
|---|---|---|---|
| | Initial | after 50 | Immersion in |
| | Lubricity | Rubbings | Warm Water |
| Example 2 | OO | OO | OO |
| Comparative | x | x | x |
| Examples 1 to 4 | | | |

The surface lubricity test was conducted on the basis of feeling or touching, the surface of each test sample being touched by the finger. The case in which lubricity was good was marked “OO”, the case in which some lubricity was noted was marked “o” (in the present test, such a result was not obtained) and the case in which lubricity was poor was marked “x”. The above surface lubricity test was carried out for the initial state, after 50 rubbings and after immersion in warm water. The initial lubricity was tested by feeling with the finger a test sample that was unchanged since manufacture, and the lubricity after 50 rubbings was tested by feeling with the finger a test sample that had been wetted with physiological saline and rubbed by the finger 50 times. The lubricity after dipping in warm water was tested by feeling with the finger after the test sample had been immersed in a physiological saline of 50° C. for 24 hours.

As shown in Table 1, the result was that, in Example 2, lubricity was good in all cases while, in Comparative Examples 1 to 4, lubricity was poor in all cases. From the above, it is concluded that, when a coating on the substrate is subjected to an alkaline treatment, a good lubricity can be achieved and further that the lubricity can be maintained for long time. With regard to the antithrombotic property, the test sample of Example 2 shows the same effect, to the same extent, as the test sample of Example 1.

As mentioned above, in the medical treatment device according to the present invention, there is a coating prepared by application of a mixture solution for coating, comprising a copolymer of methyl vinyl ether with maleic acid anhydride, a quaternary ammonium salt and a polyether block amide dissolved in an organic solvent, on the surface of a substrate comprising a synthetic resin and also by impregnating urokinase and heparin therein, whereby an excellent antithrombotic property is achieved. In addition, urokinase and heparin contained in the coating are present in the coating for a long period and, therefore, the antithrombotic property is maintained for a long period. When an alkaline treatment is further applied to a predetermined part of the surface of the substrate, a good lubricity can be achieved for a long period. As a result, it is possible to produce a medical treatment device of great practical value.

Incidentally, the present invention is not limited to the above-mentioned Examples and can be modified. For example, with regard to a fibrinolysis activating enzyme, it is also possible to use streptokinase, tissue plasminogen activator, plasmin, prinolase, etc. instead of urokinase. With regard to a suppressor for suppressing blood coagulation, it is possible to use hirudin, thrombomodulin, antiplatelet substance, etc. instead of heparin. With regard to a synthetic resin comprising the substrate, it is also possible tousepolyvinyl chloride, nylon, nylonelastomer, etc. instead of polyurethane. With regard to an alkaline aqueous solution, it is also possible to use potassium hydroxide, etc. instead of sodium hydroxide.

### BRIEF DESCRIPTION OF THE DRAWING

FIG. 1 is a graph which shows the relation between thrombus formation time and rinsing period.

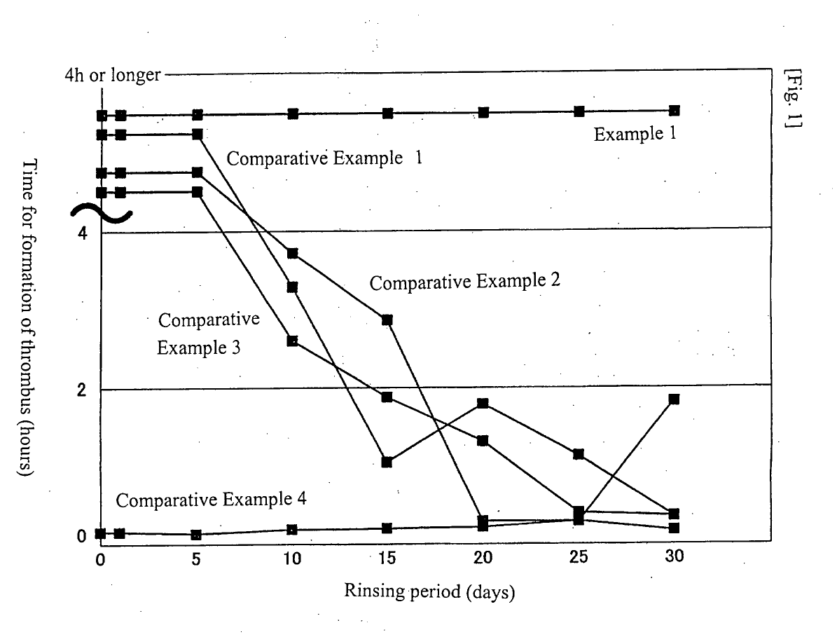

1. A medical treatment device comprising:
a substrate comprising a synthetic resin; and
a coating on at least a portion of a surface of the substrate, the coating comprising a component selected from the group consisting of fibrinolysis activating enzymes suppressors which suppress blood coagulation factor activity, and combinations thereof, in combination with a fixer selected from the group consisting of a fibrinolysis activating enzyme fixer for fixation of the fibrinolysis activating enzyme, a suppressor fixer for fixation of the suppressor which suppresses blood coagulation factor activity, a fixing reinforcer for reinforcing the fixing action of the fibrinolysis activating enzyme fixer and the suppressor fixer, and combinations thereof.

2. The medical treatment device according to claim 1, wherein a portion of the coating is subjected to an alkaline treatment by impregnating in an alkaline solution.

3. The medical treatment device according to claim 1, wherein the fixer for fibrinolysis activating enzyme comprises a copolymer of methyl vinyl ether with maleic acid anhydride, the fixer for the suppressor comprises a quaternary ammonium salts and the fixing reinforcer comprises a polyether block amide.

4. The medical treatment device according to claim 3, wherein the quaternary ammonium salt comprises tridodecylmethylammonium chloride.

5. The medical treatment device according to claim 1, wherein the fibrinolysis activating enzyme is selected from the group consisting of urokinase, streptokinase, tissue plasminogen activator, plasmin, and prinolase.

6. The medical treatment device according to claim 1, wherein the suppressor is selected from the group consisting of heparin, hirudin, thrombomodulin, and antiplatelet agents.

7. The medical treatment device according to claim 1, wherein the synthetic resin is selected from the group consisting of polyurethane, polyvinyl chloride, nylon, and of nylon elastomers.

8. A method for the manufacture of a medical treatment device comprising:
providing a medical treatment device comprising a substrate comprising a synthetic resin having a surface,
forming a solution for a coating by combining a component selected from the group consisting of fibrinolysis activating enzymes, suppressors which suppress blood coagulation factor activity, and combinations thereof, with a fixer selected from the group consisting of a fibrinolysis activating enzyme fixer for fixation of the fibrinolysis activating enzyme, a suppressor fixer for fixation of the suppressor which suppresses blood coagulation factor activity, a fixing reinforcer for reinforcing fixing action of the fibrinolysis activating enzyme fixer and the suppressor fixer, and combinations thereof, in an organic solvent to obtain a coating solution;
applying the coating solution to at least a portion of the surface of the substrate; and
obtaining a medical device possessing a coating on at least a portion of its surface.

9. The method according to claim 8, further comprising subjecting a portion of the coating to an alkaline treatment by immersion in an alkaline solution.

10. The method according to claim 8, wherein the fibrinolysis activating enzyme fixer is comprises a copolymer of methyl vinyl ether with maleic acid anhydride, the suppressor fixer comprises a quaternary ammonium salt, the fixing reinforcer for fixing action comprises a polyether block amide, the fibrinolysis activating enzyme comprises urokinase, and the suppressor is comprises heparin.

11. The method according to claim 8, further comprising drying the coating at a temperature from room temperature to 80° C.

12. The method according to claim 8, further comprising sterilizing the coating by exposing the coating to gamma radiation.

13. The method according to claim 8, wherein the quaternary ammonium salt comprises tridodecylmethylammonium chloride.

14. The method according to claim 8, wherein the fibrinolysis activating enzyme is selected from the group consisting of urokinase, streptokinase, tissue plasminogen activator, plasmin, and prinolase.

15. The method according to claim 8, wherein the suppressor is selected from the group consisting of heparin, hirudin, thrombomodulin, and antiplatelet agents.

16. The method according to claim 8, wherein the substrate comprises a synthetic resin selected from the group consisting of polyurethane, polyvinyl chloride, nylon, and nylon elastomers.

17. A method for the manufacture of a medical treatment device comprising:
providing a medical treatment device comprising a substrate having a surface, the substrate comprising a synthetic resin selected from the group consisting of polyurethane, polyvinyl chloride, nylon, and nylon elastomers;
forming a coating solution by combining, in an organic solvent,
a fibrinolysis activating enzyme selected from the group consisting of urokinase, streptokinase, tissue plasminogen activator, plasmin, and prinolase,
a fixer for fixation of the fibrinolysis activating enzyme comprising a copolymer of methyl vinyl ether with maleic acid anhydride,
a suppressor which suppresses blood coagulation factor activity selected from the group consisting of heparin, hirudin, thrombomodulin, and antiplatelet agents,
a fixer for fixation of the suppressor which suppresses blood coagulation factor activity comprising a quaternary ammonium salt, and
a fixing reinforcer comprising a polyether block amide for reinforcing the fibrinolysis activating enzyme fixer and the suppressor fixer;
applying the coating solution to at least a portion of the surface of the substrate; and
obtaining a medical device possessing a coating on at least a portion of its surface.

18. The method according to claim 17, further comprising subjecting a portion of the coating to an alkaline treatment by immersion in an alkaline solution.

19. The method according to claim 8, further comprising drying the coating at a temperature from room temperature to 80° C.

20. The method according to claim 8, further comprising sterilizing the coating by exposing the coating to gamma radiation.
